(19) United States
(12) Patent Application Publication
(10) Pub. No.: US 2010/0015669 A1
(43) Pub. Date: Jan. 21, 2010

(54) METHOD FOR CONSTRUCTING MICRORNA ADENOVIRUS EXPRESSION PLASMIDS OF SEVERE HEPATITIS RELATED HFGL2, HFAS AND HTNFR1 GENES AND PHARMACEUTICAL USE THEREOF

(76) Inventors: Qin NING, Wuhan (CN); Xiaoping LUO, Wuhan (CN); Dong XI, Wuhan (CN); Sui GAO, Wuhan (CN); Chuanlong ZHU, Wuhan (CN); Jianwen GUO, Wuhan (CN); Weiming YAN, Wuhan (CN); Ming WANG, Wuhan (CN)

Correspondence Address: MATTHIAS SCHOLL, 14781 MEMORIAL DRIVE, SUITE 1319, HOUSTON, TX, 77079, US

(21) Appl. No.: 12/269,874

(22) Filed: Nov. 12, 2008

(30) Foreign Application Priority Data
Jul. 18, 2008 (CN) 200810133445.1

(51) Int. Cl. C12N 15/66; C12N 15/64; C12N 15/63

(52) U.S. Cl. 435/91.4; 435/320.1

(57) ABSTRACT

Provided is a method for constructing microRNA adenovirus expression plasmids comprising severe hepatitis related genes of hfgl2 (Human Fibrinogen-like protein 2) prothrombinase, Fas and TNFR1 (tumor necrosis factor receptor 1), and the specificity and effectivity of microRNA interference plasmids are detected at cellular level so that a combined use of three microRNA adenovirus expression plasmids for the treatment of severe hepatitis can be achieved. According to the present invention, pAd/CMV/V5-DEST vectors and pcDNA expression plasmid of hfgl2, hFas and hTNFR1 are used to construct pAd-hfgl2, pAd-hFas and pAd-hTNFR1 by means of Gateway technology.

### CROSS-REFERENCE TO RELATED APPLICATIONS

Pursuant to 35 U.S.C. §119 and the Paris Convention Treaty, this application claims the benefit of Chinese Patent Application No. 200810133445.1 filed on Jul. 18, 2008, the contents of which are incorporated herein by reference.

### BACKGROUND OF THE INVENTION

1. Field of the Invention

The present invention relates to a method for constructing a group of gene biological agents, and more particularly to a method for constructing microRNA adenovirus expression plasmids comprising severe hepatitis related genes of hfgl2 (Human Fibrinogen-like protein 2) prothrombinase, Fas and TNFR1 (tumor necrosis factor receptor 1). Those three plasmids are used in combination to treat severe hepatitis and verify their pharmaceutical use.

2. Description of the Related Art

Due to high incidence and huge harmfulness, viral hepatitis has become a vital disease seriously impacting people's health and living standard and constraining the economic development. So far, there is no certain effective treatment for severe hepatitis. Thus, exploring the pathogenesis and control method of severe hepatitis is an important issue in the field of basic application and clinical research.

By using MHV-3 (mouse hepatitis virus-3) to infect different species of inbreeding mice, various hepatitis similar to human clinical hepatitis has been successfully established, comprising fulminant hepatitis, chronic hepatitis and clinical healthy virus vectors (Patent Application No. 02115980.7); mouse fgl2 (mfgl2) prothrombinase (fibroleukin) gene induced by the MHV-3 is highly and selectively expressed in mouse lesion liver tissues. The gene expression level is closely related to fibrin deposition in the liver tissues and extensive liver cell necrosis; when the MHV-3 infected mice are injected with mouse fgl2 mouse fgl2 antibody, the severity of liver disease can be alleviated and the morality can be significantly reduced. These results have shown that mouse fgl2 prothrombinase plays an important role in the pathogenic process of murine fulminant hepatitis. Based on these results, further researches have shown that human fgl2 (hfgl2) prothrombinase is highly expressed in Kupffer cells, vascular endothelial cells and necrosis fibrosis regions in liver tissues of severe hepatitis B patients. For viral hepatitis, fgl2 can induce fibrin deposition in liver and microcirculation obstruction in microvessel, leading to liver cell necrosis. This indicates that fgl2 prothrombinase plays a crucial role in the deterioration progress of human viral hepatitis.

Liver cell death of severe hepatitis comprises cell necrosis and apoptosis. A rapid apoptosis is one of the most important mechanisms that trigger fulminant hepatitis. Fas gene widely expressed in human tissues belongs to a tumor necrosis factor receptor super family (TNFRSF) and is named as CD95. Fas/FasL-mediated apoptosis plays an important role in infectious diseases, autoimmune diseases and cancer incidence, acting as an important link of viral hepatitis, alcoholic liver diseases, autoimmune hepatitis, acute and chronic liver failure and transplant rejection that causes inflammatory injury of liver tissues and cells. Liver cells are very sensitive to Fas-mediated apoptosis. According to literatures, due to the activation of virus antigen, Fas is expressed in liver cells and FasL is expressed in local largely-soaked CTL, leading to liver cell apoptosis. The expression level of Fas/FasL in liver cells is in accordance with disease activity, and Fas-mediated liver cell apoptosis plays an important role in liver injury.

Tumor necrosis factors (TNF) comprise TNFα and TNFβ. TNFα is mainly secreted by activated monocytes/macrophages and is a kind of peptide regulatory factors exhibiting a wide range of biological activity. TNFα plays an important role in endotoxic shock, inflammation, immune regulation, cytotoxicity and anti-virus activities. Various cell reactions induced by TNFα are achieved via two specific receptors on cell surface. The two specific receptors are 55 KD tumor necrosis factor receptor I (TNFR I) and 75 KD tumor necrosis factor receptor II (TNFR II). Many biological effects of TNFα comprising apoptosis are achieved via TNFR I. TNFR II assists signal transduction. Liver cell apoptosis caused by high expression of TNFR I plays an important role in the occurrence and development of human severe hepatitis.

In clinic, a variety of causes can result in severe hepatitis and the incidence is extremely dangerous. A large area of liver cell death can occur in patients' liver in a short time. Nowadays, there is no specific and effective treatment. Researches have shown that liver cell apoptosis mediated by Fas and TNFR I is related with liver injury of severe hepatitis. A larger number of antigen-specific cytotoxic T-lymphocytes are activated and highly concentrated in patients' liver, inducing apoptosis via Fas/FasL or TNFα/TNFR I and further destructing liver cells. For viral hepatitis, hfgl2 prothrombinase can induce fibrin deposition in liver and microcirculation obstruction in microvessel, leading to liver cell necrosis. These researches have shown that fgl2 prothrombinase, Fas/FasL and TNFα/TNFR I system play a crucial role in the deterioration progress of human viral hepatitis.

RNA interference (RNAi) is a mechanism that guides specific homologous double-stranded RNA (dsRNA) aiming at a target gene and inducing to form a RNA-induced silencing complex (RISC) to degrade target mRNA, leading to non-expression or reduced expression of the target gene. MicroRNA is a non-coding single-stranded nucleotide fragment of about 22 nt, originating from a hairpin precursor (pre-microRNA) of about 70 nt cut by a Dicer enzyme. The precursor is formed by endogenetic gene transcription. MicroRNA can bind with the untranslated area of mRNA of the target gene and inhibit mRNA translation but does not influence the stability. Just like RISC, MicroRNA can also degrade target RNA that is completely complementary with it. As a new gene interference tool, microRNA exhibits many advantages comprising high efficiency, specificity and rapidness, bringing a new perspective to the research of gene regulation and the development of gene therapy. Many international studies have shown that the interference effect of gene expression is better than antisense technology, having more clinical values.

Advantages of application of adenovirus vectors comprise:

1) a wide range of host and low pathogenicity to people;

2) infection and gene expression in non-proliferation and proliferation cell;

3) no integration into chromosomes and no mutation due to insertion;

4) homology with human genome;

5) effective proliferation and high titer;

6) simultaneous expression of multiple genes.

### SUMMARY OF THE INVENTION

In view of the above-described problems, it is one objective of the invention to provide a method for constructing miRNA expression plasmids of hfgl2 prothrombinase, hFas and hTNFR1.

It is another objective of the invention to use miRNA expression plasmids of hfgl2 prothrombinase, hFas and hTNFR1 to inhibit the expression of hfgl2 prothrombinase, hFas and hTNFR1 genes, and verify the pharmaceutical use.

To achieve the above objectives, in accordance with one embodiment of the invention, provided is a method for constructing microRNA adenovirus expression plasmids having severe hepatitis related genes of hfgl2, Fas and TNFR, the method comprising constructing pAd-hfgl2, pAd-hFas and pAd-hTNFR1 by means of Gateway technology and using pAd/CMV/V5-DEST vectors and expression plasmid pcDNA of hfgl2, hFas, hTNFR1.

In accordance with one embodiment of the invention, the construction of expression plasmid pcDNA is to respectively insert a double-stranded oligonucleotide of microRNA of hfgl2 prothrombinase, hFas and hTNFR1 into an expression vector pcDNA™ 6.2-GW/EmGFP-miR of microRNA.

In accordance with one embodiment of the invention, a single-stranded oligonucleotide sequence of miRNA against hfgl2 prothrombinase is:

5′-TGCTGTTCTTTGAACACCTCCTCGATGTTTTGGCCACTGACTGACAT
CGAGGATGTTCAAAGAA-3′

In accordance with one embodiment of the invention, a single-stranded oligonucleotide sequence of miRNA against hFas is:

5′-TGCTGTAAGTTGGAGATTCATGAGAGTTTTGGCCACTGACTGACTCT
CATGACTCCAACCTTA-3′

In accordance with one embodiment of the invention, a single-stranded oligonucleotide sequence of miRNA against hTNFR1 is:

5′-TGCTGTTCAGGTGTCGATTTCCCACAGTTTTGGCCACTGACTGACTG
TGGGAACGACACCTGAA-3′

In accordance with one embodiment of the invention, names of preserved culture of expression plasmid pcDNA are: Escherichia coli Top10/phfgl2-miRNA/Escherichia coli Top10/phTNFR1-miRNA/Escherichia coli Top10/phFas-miRNA. They are preserved in China Center for Type Culture Collection, and the accession numbers are: CCTCC NO: 207188/CCTCC NO: M207189/CCTCC NO: M207187.

In accordance with one embodiment of the invention, the method for constructing expression plasmid comprises the steps of:

1. Constructing pcDNA expression plasmid

The construction of miRNA pcDNA expression plasmid for the expression of hfgl2 prothrombinase, hFas and hTNFR1 comprises:

1) obtaining miRNA templates of hfgl2 prothrombinase, hFas and hTNFR1 genes using miRNA software of Invitrogen Corporation and synthesizing single-stranded miRNA templates;

2) inserting double-stranded miRNA templates of annealing hfgl2 prothrombinase, hFas and hTNFR1 genes into miRNA expression vectors pcDNA™ 6.2-GW/EmGFP-miR; and

3) performing sequencing and screening on miRNA expression plasmids of hfgl2 prothrombinase, hFas and hTNFR1 genes with no mutation.

2. Detecting the inhibition effect of miRNA pcDNA expression plasmid at a cellular level: respectively detecting the specific inhibition effect of miRNA expression plasmids of hfgl2 prothrombinase, hFas and hTNFR1 on the expression of hfgl2 prothrombinase, hFas and hTNFR1 genes using real-time PCR and western-blot method to respectively verify at the level of gene and protein that miRNA expression plasmids of hfgl2 prothrombinase, hFas and hTNFR1 can specifically and effectively inhibit the expression of hfgl2 prothrombinase, hFas and hTNFR1 genes in 293T cell.

3. Constructing miRNA adenovirus expression plasmid:

1) by means of Gateway technology, recombining miRNA pcDNA expression plasmid of hfgl2, hFas and hTNFR1 with pAd/CMV/V5-DEST vectors respectively to form pAd-hfgl2, pAd-hFas and pAd-hTNFR1 adenovirus expression plasmid;

2) performing sequencing and screening on pAd-hfgl2, pAd-hFas and pAd-hTNFR1 adenovirus expression plasmid with no mutation.

### BRIEF DESCRIPTION OF THE DRAWINGS

The invention is described hereinbelow with reference to accompanying drawings, in which:

FIG. 1 is a diagram illustrating interference effect of real-time quantitative PCR testing hFas-miRNA in accordance with one embodiment of the invention;

Labels:
1. blank control group (without any plasmid transfection into 293T cell);
2. positive control group (only pcDNA3.0-Fas);
3. irrelevant control group (pcDNA3.0-Fas+irrelevant miRNA);
4. experimental group 1 (pcDNA3.0-Fas+hFas-miRNA1);
5. experimental group 2 (pcDNA3.0-Fas+hFas-miRNA2);
6. experimental group 3 (pcDNA3.0-Fas+hFas-miRNA3).

FIG. 2 is a diagram illustrating interference effect of western-blot testing hFas-miRNA in accordance with one embodiment of the invention;

Labels:
1. blank control group (without any plasmid transfection into 293T cell);
2. positive control group (only pcDNA3.0-Fas transfection);
3. irrelevant control group (pcDNA3.0-Fas+irrelevant miRNA);
4. experimental group1 (pcDNA3.0-Fas+hFas-miRNA1);
5. experimental group2 (pcDNA3.0-Fas+hFas-miRNA2);
6. experimental group3 (pcDNA3.0-Fas+hFas-miRNA3).

FIG. 3 is a diagram illustrating interference effect of real-time quantitative PCR testing hfgl2-miRNA in accordance with one embodiment of the invention;

Labels:
1. positive control group (pcDNA3.1-hfgl2);
2. experimental group1 (phfgl2-miRNA1+PcDNA3.1-hfgl2);
3. experimental group2 (phfgl2-miRNA2+PcDNA3.1-hfgl2);
4. experimental group3 (phfgl2-miRNA3+PcDNA3.1-hfgl2);
5. irrelevant control group (irrelevant miRNA+PcDNA3.1-hfgl2);
6. blank control.

FIG. 4 is a diagram illustrating interference effect of western-blot testing hfgl2-miRNA in accordance with one embodiment of the invention;

Labels:
M: Marker; B: Blank;
1. experimental group 1 (phfgl2-miRNA1+PcDNA3.1-hfgl2);
2. experimental group 2 (phfgl2-miRNA2+PcDNA3.1-hfgl2);
3. experimental group 3 (phfgl2-miRNA3+PcDNA3.1-hfgl2);
4. irrelevant control group (irrelevant miRNA+PcDNA3.1-hfgl2);
5. positive control group (pcDNA3.1-hfgl2).

FIG. 5 is a diagram illustrating interference effect of real-time quantitative PCR testing

hTNFR1-miRNA in accordance with one embodiment of the invention;

Labels:
1. blank control group (without any plasmid transfection into 293T cell);
2. positive control group (only pcDNA3.0-TNFR1);
3. irrelevant control group (pcDNA3.0-TNFR1+irrelevant miRNA);
4. experimental group1 (pcDNA3.0-Fas+TNFR1-miRNA1);
5. experimental group2 (pcDNA3.0-Fas+TNFR1-miRNA2);
6. experimental group3 (pcDNA3.0-Fas+TNFR1-miRNA3).

### DETAILED DESCRIPTION OF THE INVENTION

According to one embodiment of the invention, provided is a method for constructing miRNA expression plasmids of hfgl2 prothrombinase, hFas and hTNFR1 and verifying an interference effect at a cellular level, the method comprising the steps of (taking the construction of hFas-microRNA expression plasmid as an example):

1. Constructing pcDNA Expression Plasmid

Constructing hFas-miRNA expression plasmid: 1) according to an hFas cDNA sequence on PubMed, designing and synthesizing three pairs of single-stranded complementary oligonucleotides using miRNA software of Invitrogen Corporation; 2) according to a molecular cloning operation, inserting ds Oligo into miRNA expression plasmid pcDNA™ 6.2-GW/EmGFP-miR to construct three hFas-miRNA plasmids: p-hFasmiRNA1, p-hFasmiRNA2 and p-hFasmiRNA3; 3) performing sequencing and screening on miRNA expression plasmids of hFas gene with no mutation.

2. Detecting the Inhibition Effect of miRNA pcDNA Expression Plasmid at Cellular Level

Nowadays, there is nodirect evidence of related experimental methods about whether microRNA can specifically bind to target genes and effectively express after introduction into the human body. The interference effect of these biological agents can only be reflected indirectly through the interference experiments in vitro. The inventor constructs miRNA expression plasmids of hfgl2 prothrombinase, hFas and hTNFR1, which are studied through the experiments at cellular level. The processes are described below (taking the inhibition in vitro of hFas-miRNA expression plasmid on the expression of Fas gene in 293T cell as an example):

Materials:

miRNA expression plasmid: p-hFasmiRNA1, p-hFasmiRNA2 and p-hFasmiRNA3, expressing hFas-miRNA so as to inhibit the expression of hFas gene.

Irrelevant Control Plasmid:

pcDNA3.0-hFas: hFas eukaryotic gene expression vectors, being introduced into Fas gene through transfection for exogenous expression in cell line.

293T Cell: Human Embryonic Kidney Cell

Method:

In an experimental group, using lipofectamine2000, 293T cells are cotransfected by p-hFasmiRNA1, p-hFasmiRNA2 and p-hFasmiRNA3 combined with pcDNA3.0-hFas respectively; in a blank control group, no plasmid is transfected; in positive control group, only pcDNA3.0-hFas is transfected; cotransfected irrelevant control and pcDNA3.0-hFas are treated as irrelevant control group. Cell RNA is extracted after 48h's transfection and the mRNA expression of Fas genes is detected using real-time PCR. Subsequently cells are lysed and proteins are extracted, and the expression of Fas proteins is detected using western-blot.

Results:

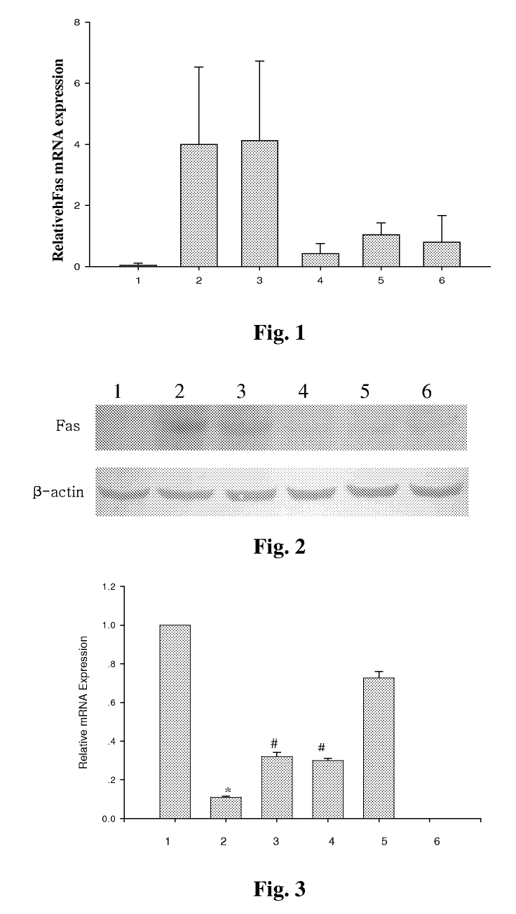

According to the detection of real-time PCR and western-blot, p-hFasmiRNA1, p-hFasmiRNA2 and p-hFasmiRNA3 can specifically inhibit hFas expression at the level of gene (FIG. 1) and protein (FIG. 2).

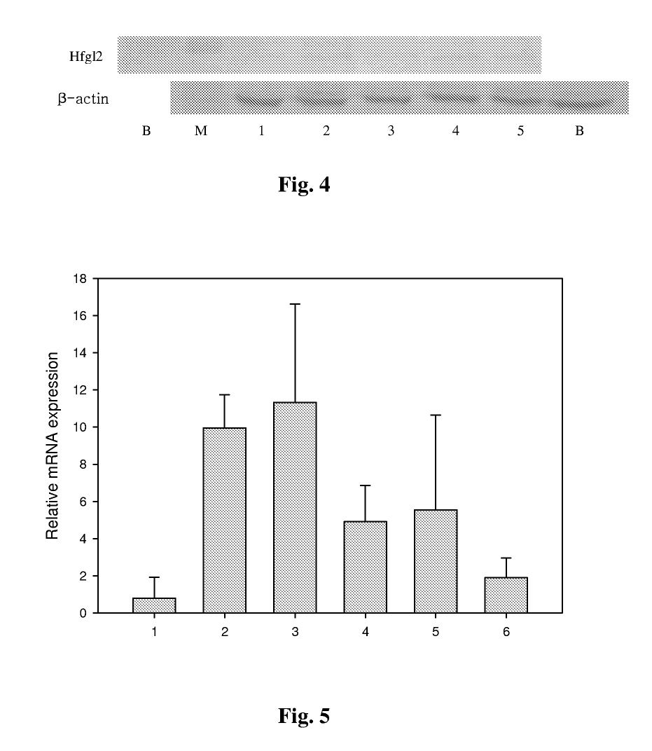

A series of experiments in vitro have shown that the new biological agents of miRNA expression plasmid of hfgl2 prothrombinase and hTNFR1, can effectively interfere in the expression of corresponding target genes (FIG. 3, 4, 5). After 48 h transfection on 293T cell line, the inhibition rates of p-hfgl2 miRNA to hfgl2, p-hFas miRNA to hFas, p-hTNFRI miRNA to hTNFRI at the level of RNA are respectively 89.3%, 87.5% and 80%. The combined use of miRNA expression plasmids also shows similar and good interference effect. For the treatment of severe hepatitis, especially the improvement of severe hepatitis patients' survival rate and the extension of graft survival time, the present invention exhibits profound and important pharmaceutical significance.

3. Constructing miRNA Adenovirus Expression Plasmid:

1) by means of Gateway technology, recombining miRNA pcDNA expression plasmid of hfgl2, hFas and hTNFR1 with pAd/CMV/V5-DEST vectors respectively to form pAd-hfgl2, pAd-hFas and pAd-hTNFR1 adenovirus expression plasmid;

2) performing sequencing and screening on pAd-hfgl2, pAd-hFas and pAd-hTNFR1 adenovirus expression plasmid with no mutation.

While particular embodiments of the invention have been shown and described, it will be obvious to those skilled in the art that changes and modifications may be made without departing from the invention in its broader aspects, and therefore, the aim in the appended claims is to cover all such changes and modifications as fall within the true spirit and scope of the invention.

The invention claimed is:

1. A method for constructing microRNA adenovirus expression plasmids of severe hepatitis related hfgl2, hFas and hTNFR1 genes, comprising constructing pAd-hfgl2, pAd-hFas and pAd-hTNFR1 by means of Gateway technology and using pAd/CMV/V5-DEST vectors and pcDNA expression plasmid of hfgl2, hFas, hTNFR1.

2. The method for constructing microRNA adenovirus expression plasmids of severe hepatitis related hfgl2, hFas and hTNFR1 genes of claim 1, wherein the construction of pcDNA expression plasmid is to respectively insert the double-stranded oligonucleotides of microRNA of hfgl2 prothrombinase, hFas and hTNFR1 into the expression vectors pcDNA™ 6.2-GW/EmGFP-miR of microRNA.

3. The method for constructing microRNA adenovirus expression plasmid of severe hepatitis related hfgl2, hFas and hTNFR1 genes of claim 2, wherein a single-stranded oligonucleotide sequence of miRNA against hfgl2 prothrombinase is:
5′-TGCTGTTCTTTGAACACCTCCTCGATGTTTTGGCCACTGACTGACAT
CGAGGATGTTCAAAGAA-3′

4. The method for constructing microRNA adenovirus expression plasmid of severe hepatitis related hfgl2, hFas and hTNFR1 genes of claim 2, wherein a single-stranded oligonucleotide sequence of miRNA against hFas is:
5′-TGCTGTAAGTTGGAGATTCATGAGAGTTTTGGCCACTGACTGACTCT
CATGACTCCAACCTTA-3′

5. The method for constructing microRNA adenovirus expression plasmid of severe hepatitis related hfgl2, hFas and hTNFR1 genes of claim 2, wherein a single-stranded oligonucleotide sequence of miRNA against hTNFR1 is:
5′-TGCTGTTCAGGTGTCGATTTCCCACAGTTTTGGCCACTGACTGACTG
TGGGAACGACACCTGAA-3′

6. Names of preserved culture of pcDNA expression plasmid of claim 1 being: Escherichia coli Top10/phfgl2-miRNA/Escherichia coli Top10/phTNFR1-miRNA/Escherichia coli Top10/phFas-miRNA, these cultures being preserved in China Center for Type Culture Collection and the accession numbers being: CCTCC NO: 207188/CCTCC NO: M207189/CCTCC NO: M207187.

7. Names of preserved culture of pcDNA expression plasmid of claim 2 are: Escherichia coli Top10/phfgl2-miRNA/Escherichia coli Top10/phTNFR1 -miRNA/Escherichia coli Top10/phFas-miRNA, these cultures being preserved in China Center for Type Culture Collection and an accession number being: CCTCC NO: 207188/CCTCC NO: M207189/CCTCC NO: M207187.


SEQUENCE LISTING (3 sequences)

SEQ ID NO: 1 - DNA, 64 bp, Homo sapiens
tgctgttctttgaacacctcctcgatgttttggccactgactgacatcgaggatgttcaaagaa

SEQ ID NO: 2 - DNA, 63 bp, Homo sapiens
tgctgtaagttggagattcatgagagttttggccactgactgactctcatgactccaacctta

SEQ ID NO: 3 - DNA, 64 bp, Homo sapiens
tgctgttcaggtgtcgatttcccacagttttggccactgactgactgtgggaacgacacctgaa